(19) United States
(12) Patent Application Publication
(10) Pub. No.: US 2012/0035446 A1
(43) Pub. Date: Feb. 9, 2012

(54) METHOD AND APPARATUS FOR MONITORING DIABETIC PATIENTS

(76) Inventors: David HOLD, Aventura, FL (US); Christina MENDEZ, Fort Lauderdale, FL (US)

(21) Appl. No.: 13/033,116

(22) Filed: Feb. 23, 2011

Related U.S. Application Data
(60) Provisional application No. 61/370,862, filed on Aug. 5, 2010.

(51) Int. Cl. A61B 5/145

(52) U.S. Cl. 600/365

(57) ABSTRACT

Diabetic patients are provided with blood sugar testers that are used to check the sugar level of the patients at home. The blood sugar data from the testers is automatically transmitted to a data bank that collects the data for each day from a plurality of patients and presents the data to the physician as required. Patients may also be provided with A1c testers that measure and transmit to the data bank A1c data collected monthly. This A1c data is also presented to the physicians so that the physicians can make an appropriate drug dosage level for each patient.

### RELATED APPLICATIONS

This application claims priority to U.S. Provisional application Ser. No. 61/370,862 filed Aug. 5, 2010 and incorporated herein by reference in its entirety. The subject matter of this application is also related to application Ser. No. 13/004,267 filed Jan. 12, 2011, claiming priority to provisional application Ser. No. 61/294,154 filed Jan. 11, 2010, incorporated herein by reference.

### BACKGROUND OF THE INVENTION

A. Field of Invention

This invention pertains to a method and apparatus for monitoring diabetic patients who are treated with insulin and/or other drugs. In the present invention, the results of the tests are automatically recorded, uploaded to a data base and shared with health care providers.

B. Description of the Prior Art

Diabetes is a major disease that affects many patients throughout the world. The disease pertains to the lack of a patient's body to either manufacture adequate supply and/or fails to utilize insulin in it metabolism. At present there is no cure for the disease but it can be manage thus allowing a patient a better quality of life. This management includes testing the blood sugar of the patient one or more times a day and taking insulin and/or other drugs. (The term “taking” is Used herein generically to any act of administering a drug to a patient, including administration of the drug by taking the drug orally, by injection, etc., and administering the drug by the patient or another person). Often both the testing and the administering are done by the patient himself.

The patient then visits his physician at somewhat regular intervals and gives the physician a list of the days on which his blood sugar was measured, the data obtained and the amount of medicine that the patient took. The physician then analyzes this data and modifies the amount of insulin/drugs that the patient has to take in the future based on this analysis.

While somewhat successful, this process is not very desirable since it is relies too much on the patient. The patient first must remember to measure the blood sugar level, which is already a daunting process since it involves taking a sharp object pricking the patient's finger or other member and then applying a drop of blood on a strip. Next, the strip is inserted into a glucose meter. Next, the glucose meter is read and the data is recorded together with the date (and, optionally, the time of day) for each reading. These are already difficult tasks to be performed routinely, especially in elder patients—a large number of the diabetic population.

Moreover, recently some reports have estimated that the data obtained this way is wrong 70% of the time and, in order to correct this problem a secondary test has been suggested as well. This second test involves having a patient go every three months to a lab or other similar facility and have a separate blood test done to determine their HbA1c level. The results of the test are transmitted to the physician who then compares them to the data obtained from the glucose meter by the patient to determine how to modify the administration of insulin/drugs for the patient. In other words, the insulin management process is becoming more and more complicated.

### SUMMARY OF THE INVENTION

In the present invention, a system is provided wherein data from a glucose meter at the patient site is obtained and automatically uploaded to a data base. A master monitor reviews the data and collates it for the physician as needed. For example, the physician may obtain the data on a daily, weekly, monthly basis and/or during a visit by the patient. Optionally the HbA1c level in the blood is also measured at regular intervals (preferably monthly) at the patient site and data is also uploaded, stored in the data base, and reviewed by the physician to confirm the data from the glucose meter. The central monitor can also be configured to monitor the data from each patient on a regular basis and to alert the physician and/or others if abnormal glucose levels are detected.

The system and method has several advantages, as discussed below.

All patients will have mandatory monthly HbA1c testing at home. This provides a much more accurate picture of what is happening with the patient without requiring the patient to go to a separate lab.

In one embodiment, the communication channel used to collect the data is also used to (a) remind each patient to test his blood; and (b) take his medicine if no indicated. Each patient will be contacted as a reminder to complete this portion of the testing. The patient may also be asked to provide not only the test data but to confirm that he has taken his medicine (and/or has given himself on injection). This process has the added benefit of providing information to the master monitor and/or another service indicative of how much drugs the patient has, and, if necessary, to alert the physician, drug store, etc., that the patient is about to run out of a certain drug.

### BRIEF DESCRIPTION OF THE DRAWINGS

FIG. 1 shows a block diagram of a system configured in accordance with this invention;

FIG. 1A shows a diagrammatic view of a device used to test a blood sample for two different criteria;

FIG. 2 shows a flow chart illustrating the operation of the system; and

FIG. 3 shows a more proactive method for operating the system.

### DETAILED DESCRIPTION OF THE INVENTION

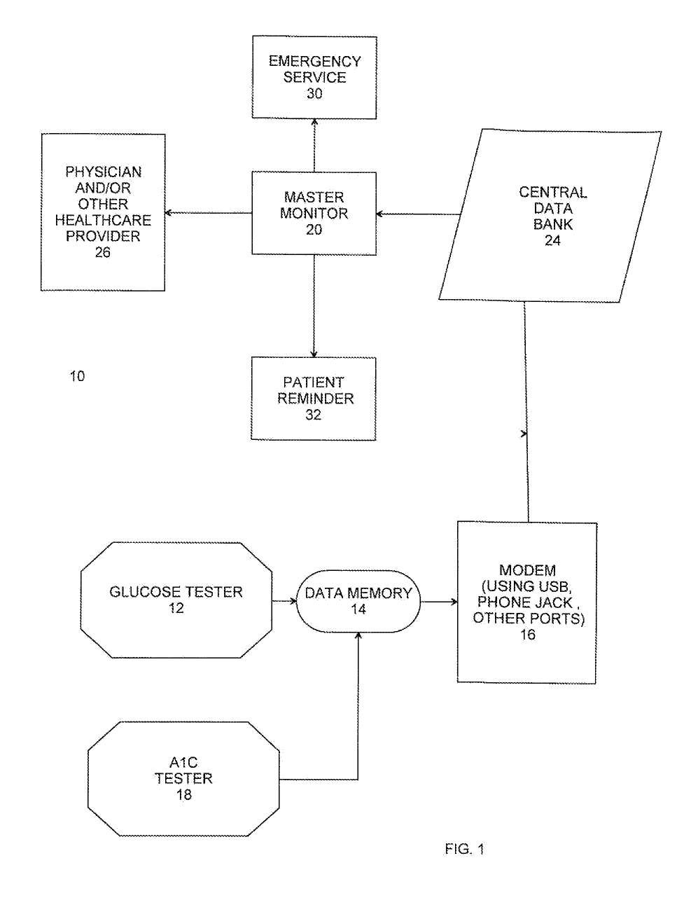

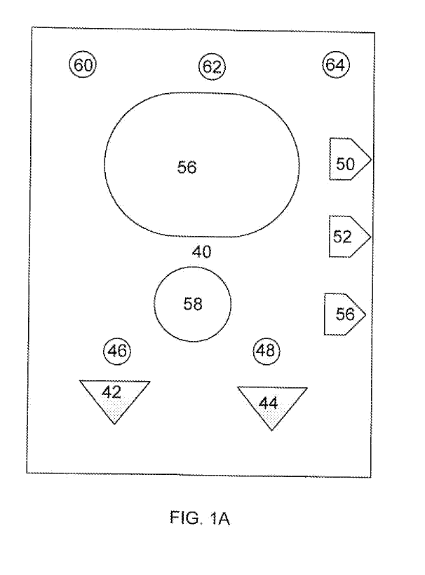

The invention is now described in conjunction with the drawings. Starting with FIG. 1, a system 10 constructed in accordance with this invention includes several elements. Some of the elements are at a patient site while others are at one or more remote locations that are interconnected by the Internet, a private communication, network and could be cloud-based.

At the patient site, there is provided a glucose tester that is similar to testers available from various sources in that it is able to accept s a blood sample from the patient, either directly, or via a strip provided for this purpose. The glucose tester determines the current sugar concentration in the blood of the patient. Once this determination is made, the resulting number as well as the current date and time are stored in a data memory 14. There is further provided a modem 16 that is connected to the data memory to transmit the data recorded therein to a remote location. The modem 16 includes a USB port, a jack for a land-line telephone, a port for a cell phone connection and/or any other similar port that can be used to establish data communication with the remote location.

In addition, there is also provided a A1C tester 18. This tester 18 also receives a blood sample and determines the HbA1c level therein. Testers of this sort are presently available, for example, from Bayer, however, they merely provide a reading and do not transmit it anywhere. Instead the results are recorded manually. In the present invention, the recording from the A1c tester are also recorded in the same data memory 14 or a separate data memory and are then transmitted by modem 16 as well.

The arrangement shown and discussed so far on FIG. 1 can be implemented in a number of different configuration. In one configuration, two separate independent testers are provided, each performing its own test, data storage and modem. In another embodiment, two physically separate testers are provided that share a data memory and/or modem. In yet another embodiment, the testers 12, 18, the data memory 14 or memories and the modem 16 or modems are disposed in a single hand held unit.

In any event, the system further includes a master monitor 20 that is connected to a central data bank 22. The central data bank receives information and stores from the modem 16 via a communication channel 24, as well as similar information from other patients. The master monitor 20 sets up What information is to receive in order to establish an effective diabetes management process for each patient. As part of this process, the master monitor exchange information with several physicians, or other health care providers and/or pharmacies and hospitals, all commonly indicated in FIG. 1 by numeral 26. In addition, the master controller 20 can also provide a reminder service 28 to its patients, and if necessary, activate an emergency service 30.

FIG. 1A shows a somewhat diagrammatic representation of a device 40 used to collect data. The device includes two ports or other means 42, 44 for receiving respectively a sample of blood for testing sugar level and A1c, respectively. Once the samples are provided at the respective ports, a respective button 46, 48 is activated to indicate to the device 40 that samples are ready to be tested. The device 40 further includes several communication ports 50, 52, 54 that may be, respectively, a USB port, an Ethernet port, a phone jack/wireless access port, etc.

Once the device 40 completes its test, the result together with other information, such as the type of test, the date and the time, are shown on a screen 56, typically an LCD screen. The result is also stored in the data memory 14. The patient then activates a button 58 requesting that the information be sent to a remote location. Alternatively, the data can be sent automatically.

Device 40 is also provided with several indication lights 60, 62, 64. Light 60 may be used to alert the patient that it is time for a test an/or the administration of a drug. Light 62 may be used to indicate to the patient that the data has been correctly received and stored, or that the data seems to be wrong and the test should be repeated. Light 64 may be used for other indication, such as a reminder or a request that he should visit his physician or other health care provider, or that his medicine is about to run out and should obtain a new supply.

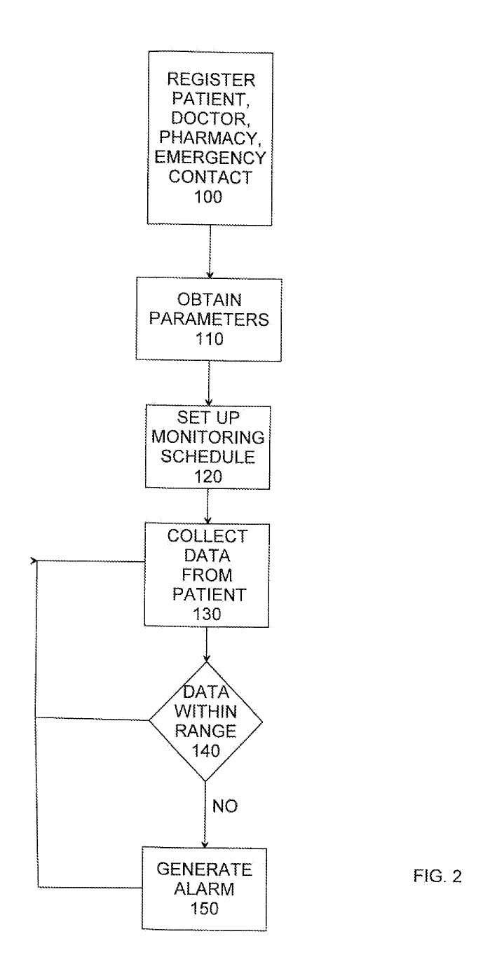

One mode of operation is now described for the system in conjunction with the flow chart of FIG. 2. In step 100 a patient registers with the system and at least some of the following information is collected. This can be done in various forms, such as having the patient fill in a form manually or electronically:
a. Name
b. Address
c. Telephone
d. E-Mail
e. Medications
f. Name of physician
g. Physician Telephone
h. Physician Address
i. Physician E-Mail
j. Escalation Notification 1
k. Escalation Notification 2
l. Escalation Notification 3
m. Any kind of authorization needed for HIPA
n. Any kind of authorization for billing insurance or Medicare/Medicaid Patients and any consent form to collect demographic information.
o. Any information for supplies and prescription from the physician in order to bill for these supplies.
p. Supply order form. Based on the amount of times the pt is testing would give the system the amount of supplies needed per month. An area on the pt form should have choices such as:
   ______ I test twice daily
   ______ I test once daily
   ______ I test once weekly

In step 110, information not received from the patient is obtained from another source, such as the physician. This information may include the amount of drugs required for the the patient, etc.

In step 120, the physician or other competent authority sets up a monitoring schedule and several levels of alarm levels for various events as defined above for j, k and l.

At the end of step 130, the central monitor 20 has only the information required for effective diabetic management process of the patient, including when the patient should receive insulin/drugs, how much he needs in a month, 3 months, etc., how often he should perform each test, including sugar tests and A1C tests (as discussed above, the sugar level typically are tested from 1 to 3 times a day, the A1c test is typically performed once a month). The management process also defines (as set by or suggested by the physician) what are the normal sugar levels, under what conditions should physicians and/or others, such as an alternate care giver, a relative or other responsible person, or, in extreme cases, an emergency service should be notified. All this information is stored in the data bank 22 and the system is now ready for operation.

In a basic mode of operation, shown in FIG. 2, staffing in step 130, data is collected from each patient device, stored in the data bank 22 and analyzed. In step 140 a determination is made if the data is within certain ranges as previously defined for each patient. If the data is within range, data collection continues in step 130. If the data exceeds a certain range or meets some other criteria, as initially defined, an alarm condition is established and data collection is continued for the other patients. Depending on preset thresholds, when an abnormal sugar level is indicated, notifications are sent automatically to the patient's physician an alternate giver, family member, emergency services, etc.

Moreover, while not shown in the flow chart, authorized personnel, including a patient's primary physician, is issued a password to allow the physician to access the patient's records at any time. For example, the physician can access the records just before, or during a standard (e.g., monthly visit), or When receiving an emergency message.

The data collection of step 130 can be implemented in various ways. For example, data can be collected asynchronously, whenever the analysis of a blood sample has been completed. Preferably, after the data is transmitted, the data bank and/or monitor analyzes the data to check if it is correct and then sends an acknowledgement that either turns on one of the indicator lights or activates a message on the screen 56 indicating that the test has been successful.

In another embodiment, the test data is stored in the memory, and the data bank poles all the data memory of the serviced patients and collects the data sequentially.

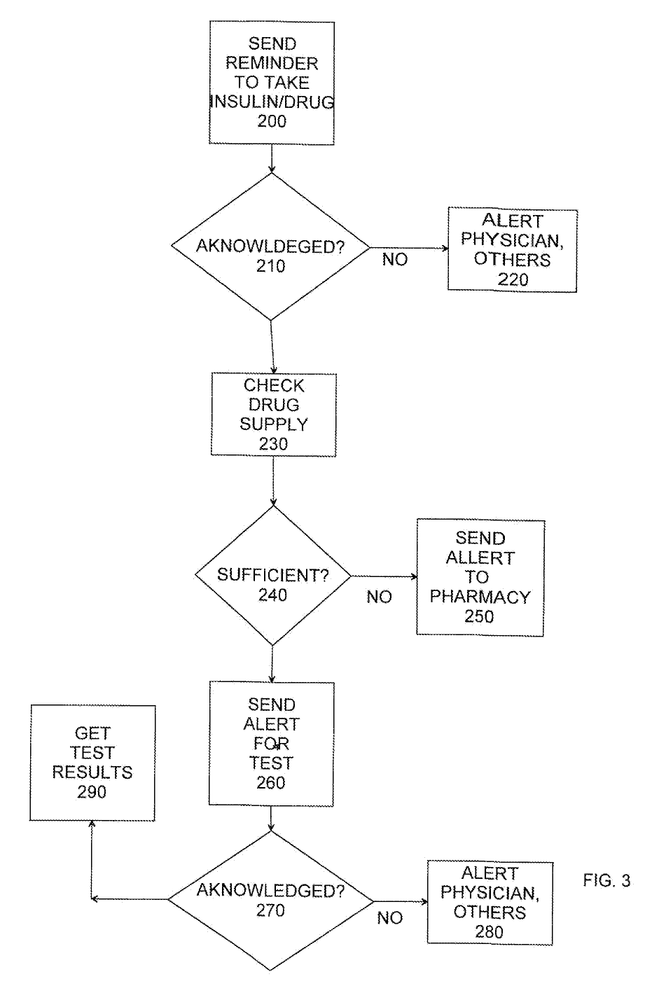

Of course, the system can be operated in another mode as well. For example, as shown in one embodiment depicted in FIG. 3, the process may start in step 200 by sending a reminder to the patient to take his medicine. This step may be implemented by either activating a light on the device (as discussed above in relation to FIG. 1C) or by other means. For example, the patient may receive an oral or text message on a land-line telephone, cell phone, e-mail, text message, etc. (A detailed description of this process is found in commonly owned co-pending application Ser. No. 13/004,267 fully identified above). In step 210, the monitor looks for an acknowledgement from the patient that he has taken the medicine or at least has received the reminder. If no acknowledgement is received, an appropriate alert is sent to the physicians, relatives or others in step 220.

If acknowledgement is received then in step 230 the monitor checks when was the last time the patient received a supply of drugs and whether that supply is getting low, the patient, or the drug store are alerted (steps 240, 250). Next in step 260 the patient is sent a reminder that he needs to test his blood sugar level. If this reminder is acknowledged (step 270, for example by pressing an appropriate button on device 40), then in step 290 the results are stored and then analyzed as previously discussed. If the reminder for a blood test is not acknowledged, an alert is sent to the physicians and/or others (step 280).

The sequence of steps in FIG. 3 are only exemplary and other sequences may be implemented as well. For example, determining how much drugs the patient should have can be made independently of the reminders. Moreover, the reminder to check for blood sugar may be before sending the reminder to administer drugs.

Many other modifications may be made to the invention without departing from its scope as defined in the appended claims.

We claim:

1. A system for treating diabetes in a patient comprising:
a glucose tester configured to receive a patient blood sample, measure the blood sugar and transmit blood sugar data electronically indicative of said blood sugar;
a data bank receiving and storing said blood sugar data; and
a master monitor coupled to said data bank and generating a reminder transmitted electronically to said patient to use said glucose tester.

2. The system of claim 1 wherein said master monitor is configured to analyze said blood sugar data from said patient and to generate a blood sugar alert based on a set of predetermined rules.

3. The system of claim 1 wherein said master monitor is configured to generate a schedule for said reminders based on patient information.

4. The system of claim 1 wherein said master monitor is configured to determine a drug supply of said patient and to generate a drug supply alert when said drug supply falls below a threshold.

5. The system of claim 1 further comprising an Al c tester for testing a patient's A1c level.

6. The system of claim 1 wherein said master monitor is configured to generate a first reminder to the patient to test the blood sugar and a second reminder to test the A1c level.

7. The system of claim 1 wherein said sugar blood tester and A1c tester are disposed in a single hand-held device.

8. A method of diabetes managing in patients having at least a blood sugar tester comprising the steps of:
transmitting a reminder to said patients to test their blood sugar level using said testers;
collecting blood sugar data from said patients; and
generating an alert when said blood sugar data from at least one of said patients meets a predetermined criteria.

9. The method of claim 8 wherein said reminder is sent to said patients based on a schedule determined by each patient's physician.

10. The method of claim 8 wherein said reminder is sent at least daily.

11. The method of claim 8 wherein at least some patients also have an A1c tester further comprising sending to patients a separate reminder to test their A1c in their blood.

12. The method of claim 11 wherein said separate reminder is transmitted monthly.

13. The method of claim 12 further comprising receiving separate data indicating some patients' A1c level in their blood.

14. The method of claim 12 wherein all data for each patient is presented to a physician.

15. The method of claim 14 further comprising determining a level of drug required by each patient based on data from said testers.

16. The method of claim 15 further comprising calculating the amount of drugs used by at least some patients and generating a drug alert when the amount left for some patients is below a threshold.
